(19) United States
(12) Patent Application Publication
(10) Pub. No.: US 2017/0011673 A1
(43) Pub. Date: Jan. 12, 2017

(54) Video Scent Display

(71) Applicant: Promo Print Solutions, Inc., Oshkosh, WI (US)

(72) Inventor: Paula Condor, Oshkosh, WI (US)

(21) Appl. No.: 14/793,897

(22) Filed: Jul. 8, 2015

(51) Int. Cl. G09F 27/00; H04N 5/645; A61L 9/12

(52) U.S. Cl. CPC G09F 27/005; A61L 9/122; H04N 5/645; G09F 2027/001

(57) ABSTRACT

A product marketing device including a housing and a scent diffuser at least partially disposed within the housing such that the scent diffuser is configured to produce an aroma of a product. A display is coupled to the housing and includes a memory and a driver and capable of displaying video data. A controller is electrically coupled to the display and the scent diffuser and the controller is capable of activating the display and activating the scent diffuser.

### BACKGROUND

The present invention relates to product marketing devices, and particularly to point of purchase product information displays and advertisements.

### SUMMARY

In one embodiment, the invention provides a product marketing device including a housing and a scent diffuser at least partially disposed within the housing such that the scent diffuser is configured to produce an aroma of a product. A display is coupled to the housing and includes a memory and a driver and capable of displaying video data. A controller is electrically coupled to the display and the scent diffuser and the controller is capable of activating the display and activating the scent diffuser.

In another embodiment the invention provides a method of advertising using a product marketing device having a housing that supports a display capable of playing video data, a scent diffuser, and a motion sensor. The method includes electrically coupling a controller to the scent diffuser, the display, and the motion sensor and placing the housing proximate a product having an aroma. The method further includes detecting with the motion sensor movement proximate the product, sending a signal from the motion sensor to the controller, and activating a driver of the display to play the video data in response to the controller receiving a signal from the motion sensor.

In yet another embodiment the invention provides a product marketing device for use in a store, including a housing having a mounting assembly for removably coupling the housing to a shelf, a scent diffuser at least partially disposed within the housing configured to produce an aroma, and a display coupled to the housing capable of displaying a video. The display includes a memory and a driver, wherein video data and audio data are stored on the memory. The device further includes a motion sensor coupled to the housing capable of sensing movement proximate the housing and a controller electrically coupled to the display, the scent diffuser, and the motion sensor. The controller is capable of activating the display via the driver upon receiving a signal from the motion sensor. A button is electrically coupled to the scent diffuser via the controller, the button capable of activating the scent diffuser when the button is operated.

Other aspects of the invention will become apparent by consideration of the detailed description and accompanying drawings.

### BRIEF DESCRIPTION OF THE DRAWINGS

FIG. 1 is an exploded view of a product marketing device.

FIG. 2 is a front view of the product marketing device of FIG. 1.

FIG. 3 is a back view of the product marketing device of FIG. 1.

FIG. 4 is a side view of the product marketing device of FIG. 1.

FIG. 5 is a cross-sectional view of the product marketing device of FIG. 1 along 4-4.

FIG. 6 is an exploded view of an internal housing of the product marketing device of FIG. 1.

FIG. 7 is a perspective back view of the internal housing shown in FIG. 6.

FIG. 8 is a back view of the internal housing.

FIG. 9 is a front view of the internal housing shown in FIG. 6.

FIG. 10 is a top view of the internal housing shown in FIG. 6.

FIG. 11 is an exploded view of a faceplate assembly of the product marketing device.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Also, it is to be understood that a plurality of hardware and software based devices, as well as a plurality of different structural components may be utilized to implement aspects of the invention. For example, the terms “mounting assembly”, “fasteners”, and variations thereof as used herein is meant to encompass equivalents thereof. Furthermore, specific configurations described or illustrated in the drawings are intended to exemplify independent embodiments of the invention and that other alternative configurations are possible. The terms “controller”, “printed circuit board”, and “PCB” and are interchangeable unless otherwise stated. Where the terms “controller”, “printed circuit board”, and “PCB” are used as identifying a unit performing specific functions, it should be understood that, unless otherwise stated, those functions can be carried out by a single controller or multiple controllers arranged in any form.

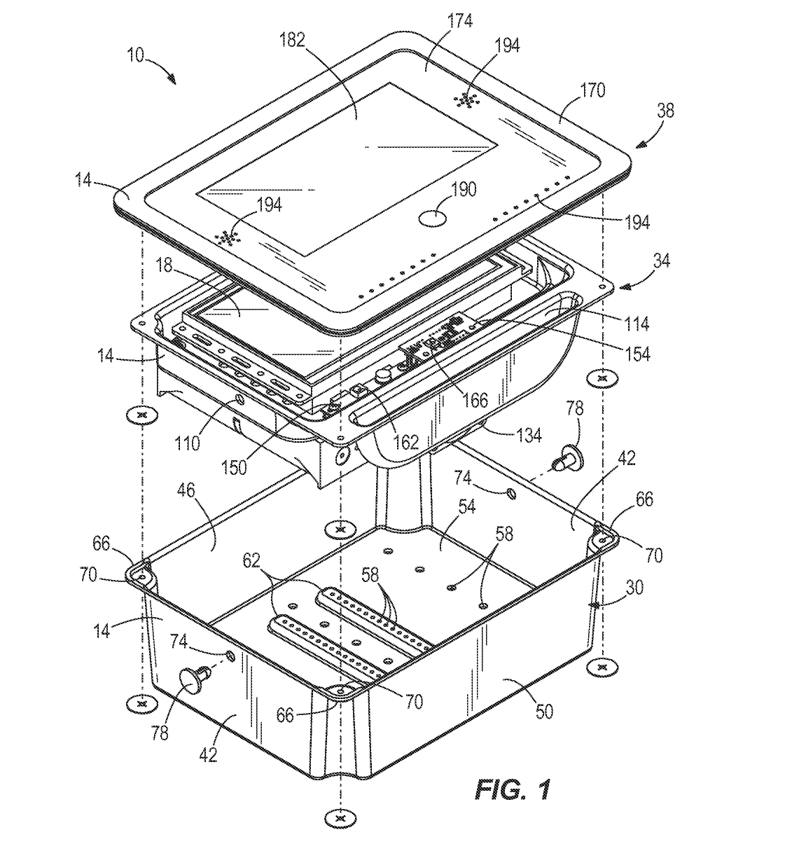

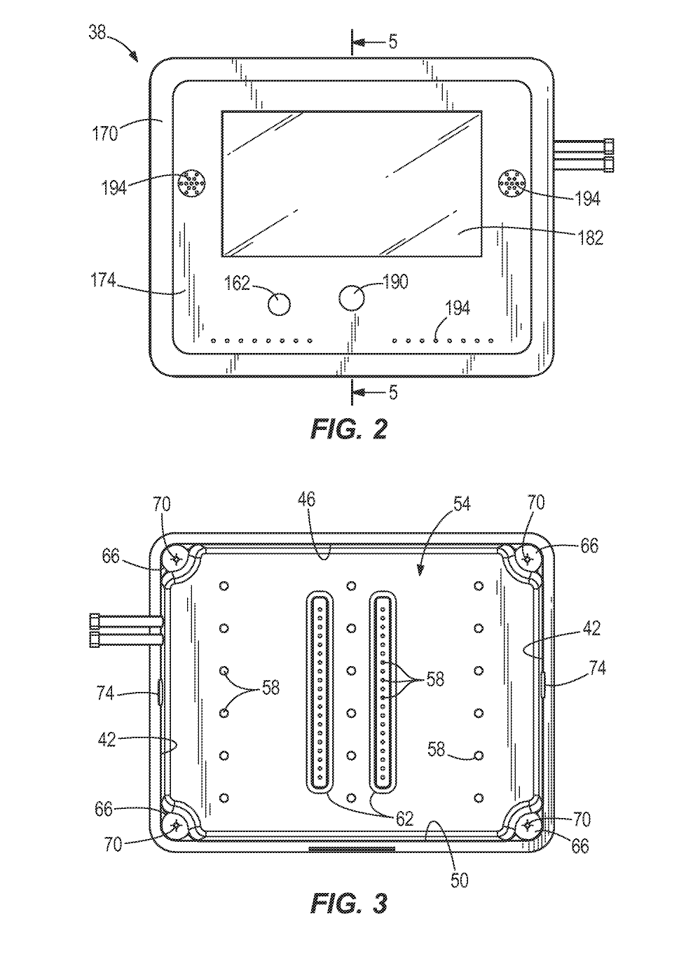

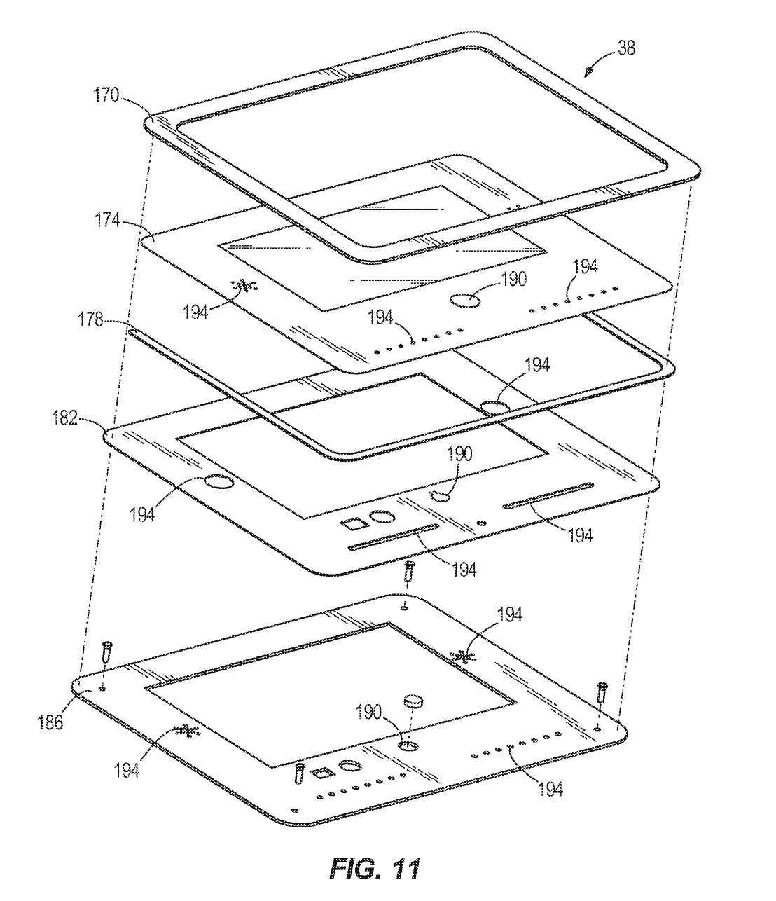

A product marketing device 10 is illustrated in FIGS. 1-11. The product marketing device 10 is designed to be used in stores and other venues where products are being sold to aid in the marketing of that product. The device 10 can be placed proximate to a product being advertised to provide information about the product to a consumer. For example, the device 10 can be used at the point of purchase, on shelves in the aisle of a store, near a check-out counter, or any other suitable advertising location. The product marketing device 10 is particularly suited to advertise products having an aroma. A consumer can engage with the product marketing device 10 and will be exposed to an advertisement for the product as well as the aroma of the product. The advertisement can include video, audio, photographs (or still images), or any combination thereof. Examples of the types of products that can be advertised on the product marketing device include, but are not limited to, cleaning products, candles and air fresheners, shampoos and lotions, food items, and perfumes.

The product marking device 10 includes a housing 14, a display 18, scent diffuser 22, and a controller 26. As illustrated in FIG. 1, the housing 14 is assembled with three portions that are fastened together including an external housing 30, an internal housing 34, and a faceplate assembly 38. FIGS. 1-5 illustrate the product marketing device 10 with the external housing 30 included. The external housing 30 includes two opposing side walls 42, a top wall 46, a bottom wall 50, and a back wall 54. A plurality of holes 58 and grooves 62 are arranged along the back wall 54 to receive one or more mounting assemblies (note shown) that enable the device 10 to be removably mounted on a shelf or wall. The plurality of holes 58 have different diameters and are spaced apart at different distances to accommodate multiple types of mounting assemblies. A tab 66 is formed at each corner of the external housing 30 where the side walls 42 meet the top wall 46 and bottom wall 50. The tabs 66 include apertures 70 for receiving fasteners (not shown) that removably coupled the external housing 30 to the internal housing 34 and the faceplate assembly 38. In addition, the side walls 42 each have one or more rivet apertures 74 for receiving a rivet 78 that removably couples the external housing 30 to the internal housing 34. The fasteners and rivets can be replaced with other fasteners known in the art.

The internal housing 34 is positioned between the external housing 30 and the faceplate assembly 38. When the device 10 is assembled, the internal housing 34 fits within the external housing 30. The internal housing 34 is provided with cavities and recesses of different sizes and shapes to accommodate the working parts of the device 10, such as the display 18, the scent diffuser 22, and the controller 26. The external housing 30 acts as a cover to protect the working parts of the device 10. A main cavity 82 of the internal housing 34 includes two opposing side walls 86, a top wall 90, a bottom wall 94, and a back wall 98. A tab 102 having an aperture 106 is formed at each corner of the internal housing 34 where the side walls 86 meet the top wall 90 and bottom wall 94. The tabs 102 of the internal housing 34 align with the tabs 66 of the external housing 30. Additionally, the side walls 86 of the internal housing 34 have rivet apertures 110 that align with the rivet apertures 74 of the external housing 30. The rivet 78 extends through the apertures 74, 110 in the sides of both the internal 34 and external housing 30. Likewise, fasteners extend through the apertures 70, 106 in the tabs 66, 102 of both the internal 34 and external housing 30.

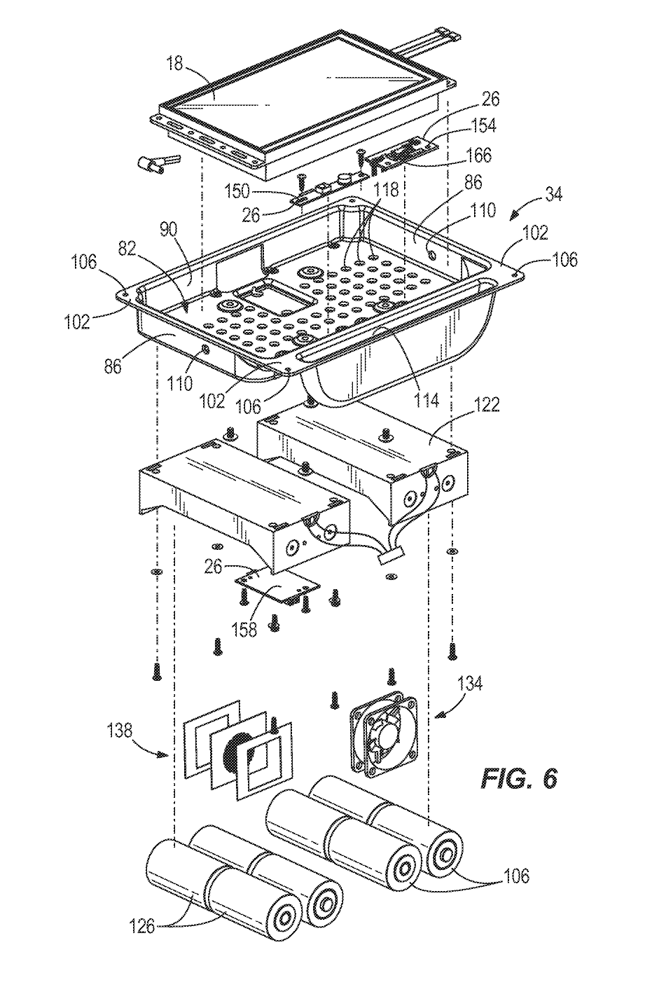

FIGS. 6-10 illustrate the product marketing device 10 with the external housing 30 removed to reveal the internal housing 34. The main cavity 82 of the internal housing 34 receives the display 18 and a portion of the controller 26. Below the main cavity 82 is an elongated recess 114 adapted for use with the scent diffuser 22. Other configurations of the recesses and cavities of the internal housing 34 are possible. For example, the recess 114 can be located to the side of the main cavity 82 or above the main cavity 82.

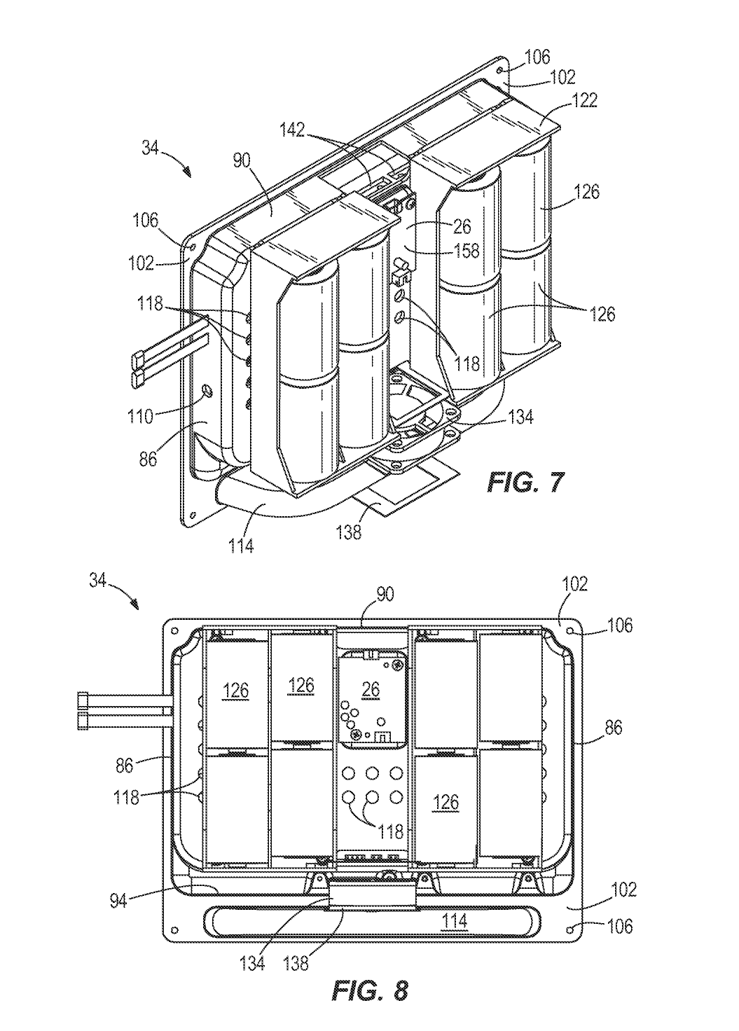

The internal housing 34 also includes a plurality of mounting holes 118 in a grid-like pattern along a back wall 98 of the main cavity 82. The plurality of holes 118 enables other working parts to be coupled to the internal housing 34 by fasteners that can be received by the holes 118. One or more battery packs 122, each containing one or more batteries 126, are coupled to the back wall 98 of the internal housing 34. As illustrated in FIGS. 7-8, the device 10 includes two battery packs 122, each containing four batteries 126. However, the number of battery packs 122, and the type of batteries 126 can vary. For example, any type of DC voltage battery (e.g., 12 volts, 18 volts, 24 volts, or 28 volts), lithium-ion battery, or rechargeable battery may be used. The number and type of batteries used will affect how frequently the batteries need to be replaced as well as the size and weight of the device 10. The size and shape of the internal 34 and external housing 30 can be adjusted to accommodate different size batteries 126. In a preferred embodiment, the device 10 is powered by eight D-cell batteries. Alternatively, AC power may be used in place of, or in addition to, DC power as is known in the art. The batteries 126 are used to power the scent diffuser 22, the display 18, and the controller 26.

With reference to FIG. 5, the scent diffuser 22 includes an aroma source 130 of a product and a fan 134. The aroma 130 is affiliated with a product located proximate the marketing device 10 for the purpose of marketing that product. The aroma source 130 can include scent beads (also denoted as 130) or other aromatic materials that can be positioned within the elongated recess 114 of the internal housing 34. For example, the aroma source can include essential oils or aromatic wax. The fan 134 is coupled to the internal housing 34 adjacent an opening (not shown) in the elongated cavity 114. A fan screen 138 is placed between the fan 134 and the opening to prevent scent beads 130 from entering the fan 134. The beads can also be contained in a bag or mesh pouch to help retain the beads and allow for easy replacement of scents. The fan 134 is configured to displace air across the scent beads 130 to diffuse the aroma outside of the device 10. The elongated housing 114 may be subdivided into multiple compartments each containing a different aroma source 130. The fan 134 can then be configured to selectively displace air across a one aroma source 130 at a time. In other embodiments, the scent diffuser 22 includes a mister or spraying device to diffuse the aroma. The mister can be used in place of, or in conjunction with, the fan 134.

With reference to FIG. 6, the display 18 is coupled to the main cavity 82 of the internal housing 34. The illustrated display 18 is a liquid crystal display (LCD), although other types of displays can be used in place of the LCD. For example, the display 18 can be a light-emitting diode display (LED), a plasma display, or a three-dimensional display. The display 18 includes a memory and a driver, and is capable of displaying video and/or audio data that is stored on the memory. The display 18 also includes speakers 154 that are capable of playing audio data that is stored on the memory. Video and/or audio data can be uploaded to the memory via an import 142. The device 10 can include more than one import 142 as well as a variety of different types of imports 142. For example, the import 142 can include a USB port, a video/audio import, a blue tooth connection, other forms of short-range communications, or a combination thereof. In addition, the display 18 includes an import 142 for a subscriber identity module (SIM) card 146 that can also be used to upload video and/or audio data to the memory. The memory, the driver, the speakers, and the imports 142, 146 can be integral with the display 18 or can be separate units that are in communication with the display 18 via the controller 26.

The controller 26 is electrically coupled to the display 18 and the scent diffuser 22. The controller 26 is configured to communicate with the display 18 and the scent diffuser 22 by sending electrical signals to the display 18 and the scent diffuser 22. For example, the controller 26 can send electrical signals to activate and deactivate the display 18 and the scent diffuser 22. The controller 26 includes one or more printed circuit boards (PCB) that are electrically coupled together. Other controllers, such as processors or central processing units, can be used in place of the PCBs. The illustrated device includes three PCBs, including a sensor board 150, an activation board 154, and a main PCB 158.

FIGS. 6-7 illustrate the main PCB 158. The main PCB 158 is coupled to the back wall 98 of the internal housing 34 by the mounting holes 118. The main PCB 158 communicates with the sensor board 150, the activation board 154, the display 18, and the scent diffuser 22 to assist in the operation of the device 10. The main PCB 158 sends signals between the other PCBs (e.g. the sensor board 150 and the activation board 154) and between the working parts (e.g. the display 18 and the scent diffuser 22) of the device 10 that are capable of receiving electrical signals. The main PCB 158 can include a variety of controls for the product marketing device 10. For example, the main PCB 158 can include a volume control, a user interface (e.g. for choosing video data stored on the memory to display), a power control (e.g. on-off switch), device setting controls (e.g. for setting the default settings).

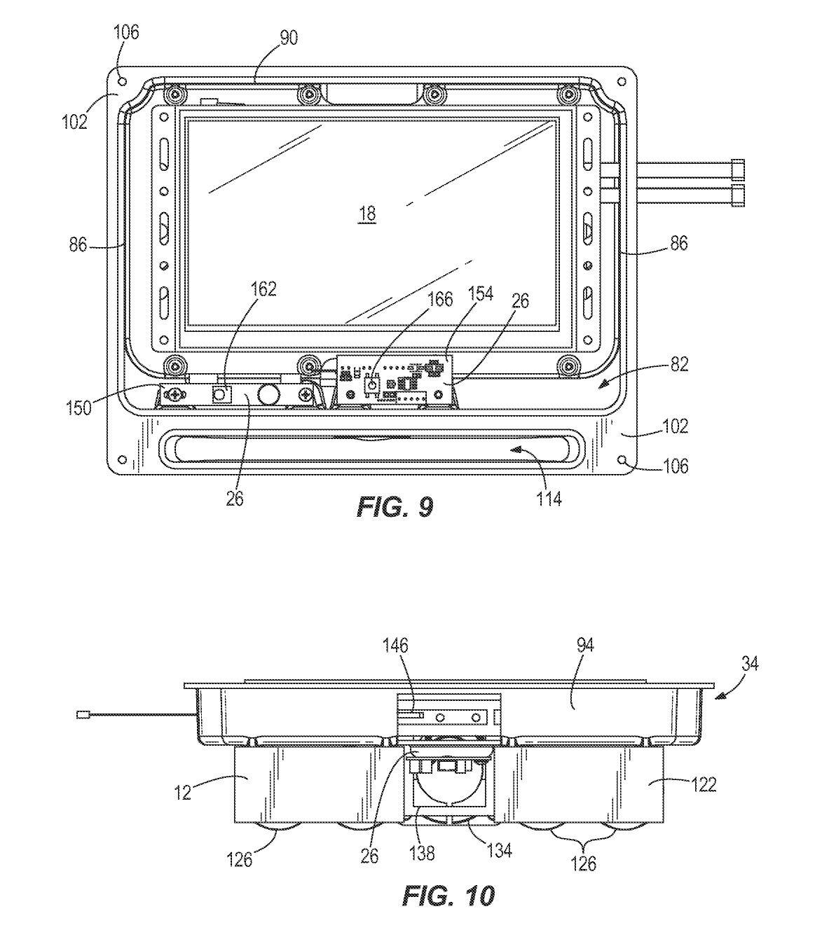

FIG. 9 illustrates the sensor board 150, which is coupled to the main cavity 82 of the internal housing 34. The sensor board 150 includes a motion sensor 162 for sensing movement proximate the device 10. Accordingly, the sensor board 150 is positioned toward a front portion of the internal housing 34 so that the sensor 162 can detect customer movement in front of the device 10. The motion detection range of the sensor 162 (i.e. the distance at which the sensor 162 is capable of detecting movement) can be varied depending upon the placement of the device 10. For example, if the device 10 placed in a store aisle, the sensor 162 can be configured to detect movement within about 5 feet of the device 10. On the other hand, if the device 10 is placed in a larger, open space, the motion detection range of the sensor 162 can be set to a greater range, for example, 15 feet from the device 10. Likewise, the motion detection angle (i.e. the angle at which the sensor 162 is capable of detecting movement) can be varied depending on the placement of the device 10. For example, if the device 10 is placed toward the end of a store aisle, the motion detection angle can be limited so that the sensor 162 can detect customer movement within the aisle, but does not detect movement of a customer that is merely walking passed the end of the aisle.

When the sensor 162 detects customer movement proximate the device 10, the sensor board 150 sends an electrical signal to the main PCB 158. In turn, the main PCB 158 sends a signal to the driver to activate the display 18 to play the video and/or audio data. In some embodiments, when the main PCB 158 receives a signal from the sensor board 150 indicating customer movement, the main PCB 158 sends a signal to the scent diffuser 22 to diffuse an aroma. The main PBC 158 can send a signal to one or both of the display 18 and the scent diffusor 22. Alternatively, the main PBC 158 can first send a signal to the display 18 to play the video, and upon reaching a predetermined point in the video, the main PBC 158 can send a delayed signal to the scent diffusor 22 to diffuse the aroma.

With continued reference to FIG. 9, the activation board 154 includes a button 166 that can be operated to activate the scent diffuser 22. More specifically, operation of the button 166 activates the fan 134, which diffuses the aroma. The button 166 can include a toggle switching, a push button, a slide button, or anything other type of actuating button or switch. When the button 166 is operated, the activation board 154 sends an electrical signal to the main PCB 158. Upon receiving the signal from the activation board 154, the main PCB 158 sends a signal to activate the fan 134. Activation of the fan 134 displaces air across the scent beads 130 to diffuse the aroma outside of the device 10.

The activation board 154, the sensor board 150, the scent diffuser 22, and the display 18 are concealed by the faceplate assembly 38. The faceplate assembly 38 is positioned on the front side of the device 10 to preferably present an advertisement of the product. The faceplate assembly 38 is removably coupled to the internal 34 and external housing 30 by fasteners that extend from the faceplate assembly 38 through the apertures 70, 102 of the internal housing 34 and external housing 30. Together the external housing 30 and the faceplate assembly 38 cover and protect the working parts of the device 10. The faceplate assembly 38 includes multiple layers of panels that stack on top of one another. Specifically, and with reference to FIG. 1, the faceplate assembly 38 includes a frame 170, a graphics panel 174, a rim 178, a clear panel 182, and a back plate 186. The panels (170, 174, 178, 182, 186) of the faceplate assembly 38 are layered on top of one another in the order mentioned such that the frame 170 is the most external panel and the back plate 186 is the most internal panel. The back plate 186 is in direct contact with the external housing 30 and the frame 170 is the most exterior panel.

Product advertisement indicia is, preferably printed on the graphic panel 174. The graphic panel 174 is able to slide between the clear panel 182 and the frame 170 so that it can be easily inserted and removed to change the advertisement indicia. Together the clear panel 182, the rim 178, and the frame 170 create a thin pocket for receiving the graphic panel 174. The rim 178 spaces apart the clear panel 182 and the frame 170 to create the pocket for the graphic panel 174 to slide into. The rim 178 is a u-shaped panel that defines an edge of the thin pocket. The u-shape of the rim 178 also enables the graphic panel 174 to be inserted from the top side, but prevents movement of the graphic panel 174 from side to side or downward. The clear panel 182 covers the display 18 to shield the display 18 from being scratched or damaged. The back plate 186 is coupled to the clear panel 182 to support the clear panel 182, the rim 178, and the frame 170. The back plate 186 is removably coupled to the external housing 30.

The back plate 186, the clear panel 182, and the graphic panel 174 includes cutouts or indentations 190 that align with the button 166 on the activation board 154 to indicate the location of the button 166 behind the panels. This configuration allows a consumer to press the button 166 through the panels. The back plate 186, the clear panel 182, and the graphic panel 184 include one or more vents 194 to allow the aroma to be diffused through the faceplate assembly 38 and out of the device 10. Likewise, the vents 194 allow the audio to be heard through the faceplate assembly 38.

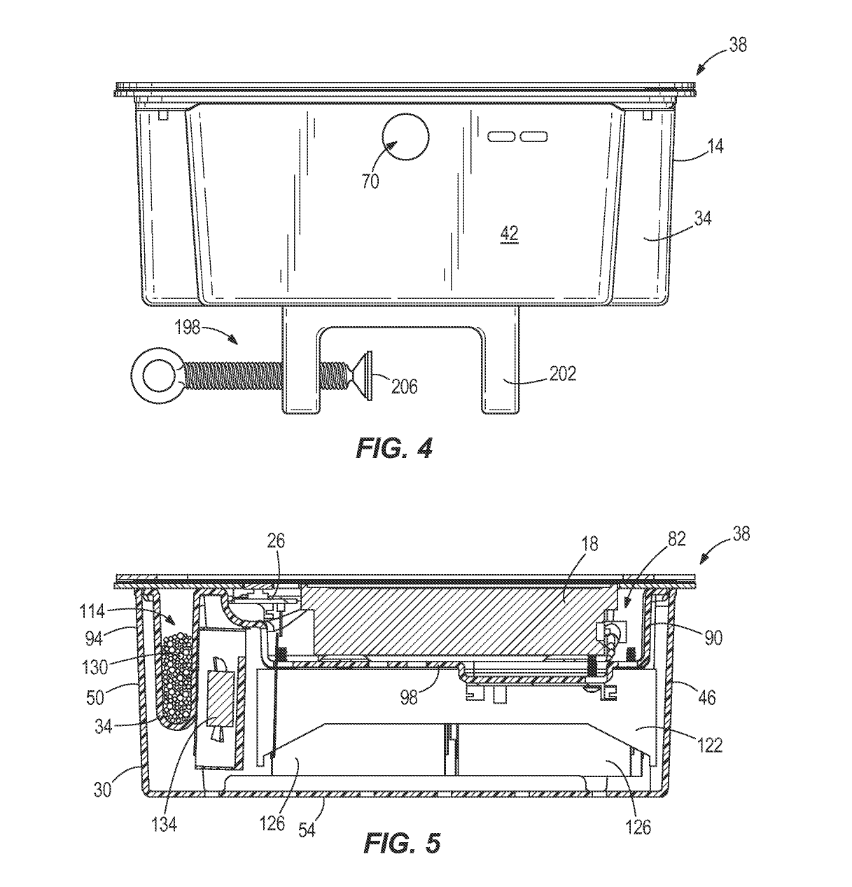

In operation, the device 10 is preferably placed near a product being advertised. The device 10 can be placed on a shelf or can be mounted to a wall or shelf by a mounting device 198. In some embodiments, the mounting device 198 is a C-clamp. As shown in FIG. 4, the C-clamp includes a C-shaped bracket 202 and a threaded clamping screw 206. Other mounting devices 198 known in the art can be used with or in place of the C-clamp. The compact size of the product marketing device 10 offers flexibility in placement within a store or other venue where products are being sold. The device 10 can be located in the aisle of a store, at the checkout counter, or any other location where a consumer can interact with the device 10.

When a consumer walks past the device 10, the motion sensor 162 detects movement in front of the device 10. In some embodiments, after movement is detected, the display 18 is activated to play the video and/or audio data to draw the attention of the consumer. More specifically, upon sensing movement, the sensor board 150 sends a signal to the controller 26 and, in turn, the controller 26 sends a signal to the display 18 to begin playing the video and/or audio data. In other embodiments, the scent diffuser 22 is activated when the sensor 162 detects movement. The motion sensor 162 can communicate with the main PCB 158 to activate the display 18, the speakers, the scent diffuser 22, or any combination thereof. The motion sensor 162 is configured to detect movement of a consumer even without the consumer noticing the marketing device 10. This enables the device 10 to engage the consumer, rather than requiring the consumer to approach the device 10 and engage with the device 10 before the device 10 becomes active.

The consumer can watch the video data being played on the display 18 to learn about the product being advertised. If desired, the consumer can operate the button 166 to diffuse the aroma so that the consumer can smell the scent of the product. Alternatively, the scent diffusor 22 can automatically diffuse the aroma at a predetermined point in the video. The products being advertised can include any product that has an aroma. For example, the product being advertised can include cleaning products, candles and air fresheners, shampoos and lotions, food items, perfumes, etc.

Additionally, the device 10 can be used to advertise more than one product at a time. Multiple videos can be stored on the memory and multiple aroma sources can be stored in the internal housing 34. Other components of the device 10 can be altered to accommodate multiple aroma sources 130. For example, additional buttons 166 can be included in the device 10 so that a consumer can choose which aroma they would like to smell. The fan 134 can be movable to direct airflow over one aroma source 130 at a time. Alternatively, multiple fans 134 can be incorporated into the device 10.

When it is desired to advertise a different product, the graphic panel 174, the aroma source 130, and the video data can be replaced. The graphic panel 174 can be replaced by simply sliding the graphic panel 174 upwards and sliding a new graphic panel 174 between the clear panel 182 and the frame 170. If a new aroma source 130 is desired, access to the elongated recess 114 containing the aroma source 130 can be acquired by removing the faceplate assembly 38 from the external housing 30. The external housing 30 can then be detached from the internal housing 34 by detaching the rivets 78 extending through the rivet apertures 74, 110 in the side walls 42, 86 of the internal 34 and external housing 30. Removal of the external housing 30 provides access to the import(s) 142 for uploading video and/or audio data. Additionally, the batteries 126 can be replaced when the external housing 30 has been removed. In some embodiments, the product marketing device 10 comes with a replacement kit including a graphic panel 174, an aroma source 130, video data, and new batteries. The replacement kits can also be purchased separately when there is a desire to market a different product.

Various features and advantages of the invention are set forth in the following claims.

1. A product marketing device comprising:
a housing;
a scent diffuser at least partially disposed within the housing, the scent diffuser configured to produce an aroma of a product;
a display coupled to the housing, the display including a memory and a driver and capable of displaying video data, and
a controller electrically coupled to the display and the scent diffuser, the controller is capable of activating the display and activating the scent diffuser.

2. The product marketing device of claim 1, further comprising a motion sensor electrically coupled to the controller for actuating at least one of the display and the scent diffuser.

3. The product marketing device of claim 2, wherein the motion sensor is configured to send a signal to the controller upon detecting movement, and wherein the controller is configured to send a command to the driver to play the video after the controller receives the signal from the motion sensor.

4. The product marketing device of claim 1, wherein the scent diffuser includes at least one set of scent beads and a fan positioned proximate the scent beads to displace air across the scent beads.

5. The product marketing device of claim 4, further comprising a button electrically coupled to the scent diffuser via the controller, wherein operation of the button activates the fan.

6. The product marketing device of claim 1, further comprising an import for uploading the video data to the display, wherein the video data is stored on the memory.

7. The product marketing device of claim 6, further comprising audio data capable of being uploaded via the import and stored on the memory.

8. The product marketing device of claim 1, further comprising a graphic panel of product information removably coupled to the housing.

9. The product marketing device of claim 1, wherein the product marketing device is powered by DC power.

10. The product marketing device of claim 1, further comprising a mounting assembly for removably coupling the housing to a shelf.

11. The product marketing device of claim 1, wherein the controller is a printed circuit board.

12. A method of advertising using a product marketing device having a housing that supports a display capable of playing video data, a scent diffuser, and a motion sensor, the method comprising:
electrically coupling a controller to the scent diffuser, the display, and the motion sensor;
placing the housing proximate a product having an aroma;
detecting with the motion sensor movement proximate the product and sending a signal from the motion sensor to the controller, and
activating a driver of the display to play the video data in response to the controller receiving a signal from the motion sensor.

13. The method of claim 12, further comprising diffusing an aroma corresponding to the product by activating the scent diffuser in response to at least one of detecting movement by the motion sensor and operating a button positioned on the housing.

14. The method of claim 13, wherein activating the scent diffuser includes operating the button positioned on the housing, the button configured to activate a fan that displaces air across a plurality of scent beads.

15. The method of claim 13, further comprising replacing the aroma corresponding to the product with a second aroma corresponding to a second product.

16. The method of claim 12, further comprising uploading video data to the display via an input and storing the video data on a memory.

17. The method of claim 16, further comprising uploading an audio file via the input and storing the audio file on the memory.

18. The method of claim 12, further comprising removably coupling a graphic panel to the housing, the graphic panel including product information.

19. The method of claim 12, further comprising powering the product marketing device by DC power.

20. The method of claim 12, further comprising automatically diffusing an aroma corresponding to the product at a predetermined point of the video data.

21. A product marketing device for use in a store, comprising:
a housing having a mounting assembly for removably coupling the housing to a shelf;
a scent diffuser at least partially disposed within the housing, the scent diffuser configured to produce an aroma;
a display coupled to the housing capable of displaying a video, the display including a memory and a driver, wherein video data and audio data are stored on the memory;
a motion sensor coupled to the housing capable of sensing movement proximate the housing;
a controller electrically coupled to the display, the scent diffuser, and the motion sensor, wherein the controller is capable of activating the display via the driver upon receiving a signal from the motion sensor; and
a button electrically coupled to the scent diffuser via the controller, the button capable of activating the scent diffuser when the button is operated.
